SEQUENCE LISTING (11 sequences)

SEQ ID NO: 1 - protein, 120 aa, Artificial Sequence
  CAR VH
EVTLVESGGGLVKPGGSLKLSCAASGFTFSTYVMSWVRQTPEKRLEWVASILSGGTSFYADNVKGRFTISRENARNILYLQMSSLKSEDTAMYYCVRRYGHDHYYAMDYWGQGTSVTVSS

SEQ ID NO: 2 - protein, 108 aa, Artificial Sequence
  CAR VL
QIVLTQSPANMSASLGERVTMTCAASSSVSSSYLHWYQQKPGSSPKLWIYSTSNLASGVPGRFNGSGSGTSYSLTISSMEAEDAATYYCHQYHRSPWTFGGGTKLEIK

SEQ ID NO: 3 - protein, 492 aa, Artificial Sequence
  Chimeric Antigen Receptor
MALPVTALLLPLALLLHAARPEVTLVESGGGLVKPGGSLKLSCAASGFTFSTYVMSWVRQTPEKRLEWVASILSGGTSFYADNVKGRFTISRENARNILYLQMSSLKSEDTAMYYCVRRYGHDHYYAMDYWGQGTSVTVSSGGGGSGGGGSGGGGSGGGGSQIVLTQSPANMSASLGERVTMTCAASSSVSSSYLHWYQQKPGSSPKLWIYSTSNLASGVPGRFNGSGSGTSYSLTISSMEAEDAATYYCHQYHRSPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 5 aa, Artificial Sequence
  Linker
GGGGS

SEQ ID NO: 5 - protein, 335 aa, Artificial Sequence
  EGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 6 - protein, 7 aa, Artificial Sequence
  VH CDR1
GFTFSTY

SEQ ID NO: 7 - protein, 5 aa, Artificial Sequence
  VH CDR2
LSGGT

SEQ ID NO: 8 - protein, 12 aa, Artificial Sequence
  VH CDR3
RYGHDHYYAMDY

SEQ ID NO: 9 - protein, 12 aa, Artificial Sequence
  VL CDR1
AASSSVSSSYLH

SEQ ID NO: 10 - protein, 7 aa, Artificial Sequence
  VL CDR2
STSNLAS

SEQ ID NO: 11 - protein, 8 aa, Artificial Sequence
  VL CDR3
QYHRSPWT

(19) United States
(12) Patent Application Publication
(10) Pub. No.: US 2025/0186492 A1
(43) Pub. Date: Jun. 12, 2025

(54) CAR-T CELL TARGETING B7-H3 AND APPLICATION THEREOF IN TREATMENT OF ACUTE MYELOID LEUKEMIA

(71) Applicant: PERSONGEN BIOTHERAPEUTICS (SUZHOU) CO., LTD., Suzhou, Jiangsu (CN)

(72) Inventors: Lin YANG, Suzhou, Jiangsu (CN); Fengtao YOU, Suzhou, Jiangsu (CN); Shuangshuang FAN, Suzhou, Jiangsu (CN); Yafen LI, Suzhou, Jiangsu (CN)

(21) Appl. No.: 18/261,410

(22) PCT Filed: Dec. 24, 2021
(86) PCT No.: PCT/CN2021/141338

§ 371 Date: Apr. 18, 2024

(30) Foreign Application Priority Data
Jan. 13, 2021 (CN) 202110044539.7

(51) Int. Cl. A61K 35/17; A61K 40/11; A61K 40/15; A61K 40/31; A61K 40/42; A61P 35/02; C07K 16/28; C12N 15/85

(52) U.S. Cl. CPC A61K 35/17; A61K 40/11; A61K 40/15; A61K 40/31; A61K 40/421; A61P 35/02; C07K 16/2827; C12N 15/85; A61K 2239/13; A61K 2239/17; A61K 2239/21; A61K 2239/25; A61K 2239/48; C07K 2317/565; C07K 2317/622

(57) ABSTRACT

The present invention provides a CAR-T cell targeting B7-H3 and an application thereof in the treatment of acute myeloid leukemia (AML). Specifically, the present invention provides a CAR-T cell targeting B7-H3, which comprises an scFv which targets B7-H3, a 41BB costimulatory signaling molecule and a CD3ζ domain. The B7-H3-CAR-T cell of the present invention has significant specific killing toward B7-H3 positive AML tumor cells. The results of animal experiments show that the B7-H3-CAR-T cell can significantly inhibit the growth of AML tumor cells in mice, significantly prolong the survival period of mice, and has a significant anti-tumor effect in vivo. The B7-H3-CAR-T cell of the present invention can be used as a novel therapeutic method for the targeted treatment of AML, and has huge clinical application prospects.

### TECHNICAL FIELD

The present invention relates to the field of biotechnology, and more particularly to a CAT-T cell targeting B7-H3 and applications thereof in treating acute myeloid leukemia.

### BACKGROUND

Acute myeloid leukemia (AML) is a prevalent type of acute leukemia. It is a highly heterogeneous malignant clonal disease that originates from the haematopoietic tissue. Patients have a high mortality rate, low long-term survival rate, and are prone to relapse, and the disease greatly endangers human health and life. For decades, there has been little change in the treatment of AML, main means for which are chemotherapy and haematopoietic stem cell transplantation. Anthracyclines combined with cytarabine remain the classic chemotherapy regimen. Despite a relatively high remission rate after treatment of such therapy, patients are at high risk of relapse because of drug resistance.

According to the AML morphological classification of French-American-British (FAB), AML can be divided into eight types: MO-M7. Studies have shown that patients with M3 and M4 subtypes have better prognosis than other subtypes, while the most common (8; 12) translocation in AML is associated with good prognosis, but rare in the M5 subtype. At the same time, there are also reports showing that patients with M5 subtype have significantly worse prognosis than other AML. Therefore, it is urgent to develop better treatment methods for AML, especially for the M5 subtype of AML.

Chimeric antigen receptor T (CAR T) cell immunotherapy induces specific activation of T cells by endowing T cells with antibodies that can recognize antigens on tumor cell surfaces and thereby avoiding the limitations of MHC. Modified T cells can better recognize and kill tumors than natural T cells. In recent years, CAR-T therapy has made great progress in cancer treatment and is considered one of the most promising tumor treatment methods.

At present, CAR T therapy has the best therapeutic effect and is most widely used in the treatment of ALL. Among them, CAR targeting CD19 has achieved significant efficacy in treating both adult and child ALL, with a remission rate of up to 90%. CAR-T treatment of AML with antigens CD123 and CD33 also demonstrated good results. However, these two antigens are expressed in normal myeloid cells, which will inevitably result in toxic side effects in patients and is still a problem that cannot be ignored at present. Therefore, finding a relatively safe and efficacious therapeutic targets is essential. Novel therapeutic targets and methods need to be developed in this field.

### SUMMARY OF THE INVENTION

The purpose of the invention is to provide a CAT-T cell targeting B7-H3 and applications thereof in treating acute myeloid leukemia.

In the first aspect of the present invention, it provides a chimeric antigen receptor (CAR), wherein an antigen binding domain (i.e. scFv) of the chimeric antigen receptor comprises a heavy chain variable region and a light chain variable region,
a CDR1 as shown in SEQ ID NO: 6,
a CDR2 as shown in SEQ ID NO: 7, and
a CDR3 as shown in SEQ ID NO: 8;
and the light chain variable region comprises the following complementarity determining regions (CDRs):
a CDR1′ as shown in SEQ ID NO: 9,
a CDR2′ as shown in SEQ ID NO: 10, and
a CDR3′ as shown in SEQ ID NO: 11.

In another preferred embodiment, the antigen binding domain of the chimeric antigen receptor comprises a heavy chain variable region of antibody as shown in SEQ ID NO: 1, and a light chain variable region of antibody as shown in SEQ ID NO: 2.

In another preferred embodiment, the heavy chain variable region of the antibody and light chain variable region of the antibody are connected with a linking peptide.

In another preferred embodiment, the structure of the antigen binding domain is shown in the following Formula I or II:

V_{L}-V_{H} (I);

V_{H}-V_{L} (II)
wherein V_{H} represents the heavy chain variable region of the antibody; VL represents the light chain variable region of the antibody; and “-” represents a linking peptide or a peptide bond.

In another preferred embodiment, the structure of the antigen binding domain is shown in Formula II (VH-VL).

In another preferred embodiment, the amino acid sequence of VH is shown in SEQ ID NO: 1, and the amino acid sequence of VL is shown in SEQ ID NO: 2.

In another preferred embodiment, the linking peptide has 1-4, preferably 2-4, more preferably 3 continuous sequences shown in SEQ ID NO: 4 (GGGGS).
in another preferred embodiment, the antigen binding domain binds to B7-H3, preferably human B7-H3.

In another preferred embodiment, the heavy chain variable region and light chain variable region of the antigen binding domain are derived from a humanized antibody.

In another preferred embodiment, the structure of the chimeric antigen receptor is shown in the following Formula III:

L-scFv-H-TM-C-CD3ζ (III)
wherein,
L is absent or a signal peptide sequence;
scFv is a scFv targeting B7-H3;
H is a hinge region;
TM is a transmembrane domain;
C is a co-stimulatory signaling molecule;
CD3ζ is a cytoplasmic signal transduction sequence derived from CD3ζ.

In another preferred embodiment, L is a signal peptide of a protein selected from the group consisting of CD8, CD28, GM-CSF, CD4, CD137 and a combination thereof.

In another preferred embodiment, L is a signal peptide derived from macrophage colony stimulating factor.

In another preferred embodiment, H is a hinge of a protein selected from the group consisting of CD8, CD28, CD137, Fc and a combination thereof.

In another preferred embodiment, H is a hinge derived from CD8.

In another preferred embodiment, TM is a transmembrane region of a protein selected from the group consisting of CD28, CD3 epsilon, CD45, CD4, CD5, CD8, CD9, CD16, CD22, CD33, CD37, CD64, CD80, CD86, CD134, CD137, CD154 and a combination thereof.

In another preferred embodiment, TM is a transmembrane region derived from CD8.

In another preferred embodiment, C is a co-stimulatory signaling molecule of a protein selected from the group consisting of OX40, CD2, CD7, CD27, CD28, CD30, CD40, CD70, CD134, 4-1BB (CD137), PD1, Dap10, CDS, ICAM-1, LFA-1 (CD11 a/CD18), ICOS (CD278), NKG2D, GITR, TLR2 and a combination thereof.

In another preferred embodiment, C is a co-stimulatory signaling molecule derived from 4-1BB.

In another preferred embodiment, the CAR further comprises a cell suicide element (preferably at C-terminus).

In another preferred embodiment, the CAR is linked with the cell suicide element via a self-cleaving element.

In another preferred embodiment, the cell suicide element is linked with CD3ζ of the CAR via a T2A.

In another preferred embodiment, the cell suicide element is selected from the group consisting of HSV-TK, iCasp9, ACD20, mTMPK, ACD19, RQR8, EGFRt and a combination thereof.

In another preferred embodiment, the cell suicide element is tEGFR.

In another preferred embodiment, the amino acid sequence of the tEGFR is shown in SEQ ID NO: 5.

In another preferred embodiment, the amino acid sequence of the CAR is shown in SEQ ID NO: 3.

In the second aspect of the present invention, it provides a nucleic acid molecule encoding the chimeric antigen receptor (CAR) according to the first aspect of the present invention.

In another preferred embodiment, the nucleic acid molecule is isolated.

In the third aspect of the present invention, it provides a vector which contains the nucleic acid molecule according to the second aspect of the present invention.

In another preferred embodiment, the vector is selected from the group consisting of DNA, RNA, plasmid, lentiviral vector, adenoviral vector, adeno-associated viral vector (AAV), retroviral vector, transposon and a combination thereof.

In another preferred embodiment, the vector is selected from the group consisting of plasmid and viral vector.

In another preferred embodiment, the vector is in the form of a viral particle.

In another preferred embodiment, the vector is a lentiviral vector.

In the fourth aspect of the present invention, it provides a host cell comprising the vector according to the third aspect of the present invention, or having the exogenous nucleic acid molecule according to the second aspect of the present invention integrated into the chromosome, or expressing the CAR according to the first aspect of the present invention.

In another preferred embodiment, the host cell includes an eukaryotic cell and prokaryotic cell.

In another preferred embodiment, the host cell includes Escherichia coli.

In the fifth aspect of the present invention, it provides an engineered immune cell expressing the CAR according to the first aspect of the present invention.

In another preferred embodiment, the cell is an isolated cell, and/or the cell is a genetically engineered cell.

In another preferred embodiment, the immune cell is derived from human or a non-human mammal (such as a mouse).

In another preferred embodiment, the cell includes a T cell and an NK cell.

In another preferred embodiment, the immune cell expresses an exogenous cell suicide element.

In another preferred embodiment, the CAR and the cell suicide element are co-expressed in the immune cell.

In another preferred embodiment, the engineered immune cell includes a T cell or an NK cell, and preferably is (i) a chimeric antigen receptor T cell (CAR-T cell); or (ii) a chimeric antigen receptor NK cell (CAR-NK cell).

In the sixth aspect of the present invention, it provides a formulation comprising the chimeric antigen receptor according to the first aspect of the present invention, the nucleic acid molecule according to the second aspect of the present invention, the vector according to the third aspect of the present invention, or the immune cell according to the fifth aspect of the present invention, and a pharmaceutically acceptable carrier.

In another preferred embodiment, the formulation is a liquid preparation.

In another preferred embodiment, the dosage form of the formulation is an injection.

In another preferred embodiment, the concentration of the CAR T cell in the formulation is 1×10³-1×10⁸ cells/ml, and preferably 1×10⁴-1×10⁷ cells/ml.

In another preferred embodiment, the formulation further comprises a second anti-tumor active ingredient, which preferably comprises a second antibody, or a chemotherapeutic agent.

In another preferred embodiment, the chemotherapeutic agent is selected from the group consisting of: Docetaxel, carboplatin, and a combination thereof.

In the seventh aspect of the present invention, it provides a use of the chimeric antigen receptor according to the first aspect of the present invention, the nucleic acid molecule according to the second aspect of the present invention, the vector according to the third aspect of the present invention, or the immune cell according to the fifth aspect of the present invention, or the formulation according to the sixth aspect of the present invention, in the preparation of a medicament or a formulation for preventing and/or treating a tumor or cancer.

In another preferred embodiment, the tumor is selected from the group consisting of: hematological tumors, solid tumors, and a combination thereof.

In another preferred embodiment, the hematological tumor is selected from the group consisting of: acute myeloid leukemia (AML), multiple myeloma (MM), chronic lymphocytic leukemia (CLL), acute lymphocytic leukemia (ALL), diffuse large B cell lymphoma (DLBCL), and a combination thereof.

In another preferred embodiment, the solid tumor is selected from the group consisting of: gastric cancer, gastric cancer peritoneal metastasis, liver cancer, leukemia, kidney tumor, lung cancer, small intestine cancer, bone cancer, prostate cancer, colorectal cancer, breast cancer, colorectal cancer, cervical cancer, ovarian cancer, lymphoma, nasopharyngeal cancer, adrenal gland tumor, bladder tumor, non-small cell lung cancer (NSCLC), brain glioma, endometrial cancer, and a combination thereof.

In another preferred embodiment, the tumor is a B7-H3-positive tumor, preferably a tumor with high expression of B7-H3.

In another preferred embodiment, the medicament or formulation is used for preventing and/or treating acute myeloid leukemia (AML).

In the eighth aspect of the present invention, it provides a kit for preparation of the host cell according to the fourth aspect of the present invention, wherein the kit comprises a container, and the nucleic acid molecule according to the second aspect of the present invention or the vector according to the third aspect of the present invention contained in the container.

In the ninth aspect of the present invention, it provides a method for preparing an engineered immune cell expressing the CAR according to the first aspect of the present invention, which comprises following steps:
(a) providing an immune cell to be modified; and
(b) transducing the nucleic acid molecule according to the second aspect of the present invention or the vector according to the third aspect of the present invention into the immune cell, thereby obtaining the engineered immune cell.

In another preferred embodiment, the engineered immune cell is a CAR-T cell or a CAR-NK cell.

In another preferred embodiment, the method further comprises a step of detecting the function and effectiveness of the obtained engineered immune cell.

In the tenth aspect of the present invention, it provides a method of treating a disease which comprises: administering an appropriate amount of the vector according to the third aspect of the present invention, or the immune cell according to the fifth aspect of the present invention, or the formulation of the sixth aspect of the present invention, to a subject in need of treatment.

In another preferred embodiment, the disease is cancer or tumor.

It should be understood that, within the scope of the present invention, the technical features specifically described above and below (such as the Examples) can be combined with each other, thereby constituting a new or preferred technical solution which needs not be described one by one.

### DESCRIPTION OF FIGURE

FIG. 1 shows the expression rate of B7-H3 antigen on the surface of AML tumor cells, wherein the expression of B7-H3 on the surface of six AML tumor cell strains are detected by flow cytometry.

FIG. 2 shows the structure and functional verification of B7-H3-CAR. FIG. 2A shows the structural schematic diagram of B7-H3-CAR which includes an EF1 promoter, a B7-H3 single chain antibody, a CD8 transmembrane domain, a co-stimulatory domain 41-BB, and an intracellular signal domain CD3ζ. FIG. 2B shows the expression of B7-H3-CAR on the surface of Jurkat cells after transfection, analyzed by flow cytometry. FIG. 2C shows the expression of CD25 and CD69 on the cell surface, wherein, Jurkat and B7-H3-CAR Jurkat cells were co-incubated with HEL, AML5, and KG-1 cells at a ratio of 1:1 for 48 hours, and the expression of CD25 and CD69 on the surface of Jurkat cells and B7-H3-CAR Jurkat cells are detected using CD25-APC and CD69-APC antibodies, respectively.

FIG. 3 shows the specific killing effect of B7-H3-CAR T cells on B7-H3⁺ AML cell lines. FIG. 3A shows the positive rate of B7-H3-CAR T cells, wherein, the positive rate of B7-H3-CAR T cells is detected as approximately 51.84% through flow cytometry analysis. FIG. 3B shows the expression of B7-H3-CAR in T cells analyzed by Western blot, wherein proteins are extracted from T (lane 1) and B7-H3-CAR T (lane 2) cells, separated by SDS-PAGE and incubated with CD3ζ chain-specific mAb and HRP-sheep-anti-mouse IgG, then detected by western blot via chemiluminescence. FIG. 3C shows the apoptotic ratio of target cells after co-incubation at a E:T ratio of 1:1 for 48 hours. ****P<0.0001, n=3. FIG. 3D shows the secretion of cytokines (IL-2, TNF, INF-γ and Grazyme B) in the supernatant after co-incubation. ****P<0.0001, ***P<0.001, **P<0.01.

FIG. 4 shows the anti-tumor activity of B7-H3-CAR T cells in AML-transplanted mice. FIG. 4A shows a schematic diagram of the AML transplantation model in NSG mice, wherein three groups of 6- to 8-week-old female NSG mice (PBS, T cell, and B7-H3-CAR-T cell groups), with 4 mice in each group, are injected with 5×10⁵ luciferase-GFP-HEL cells via the tail vein, followed by 1×10⁷ effector cells injected via the tail vein in an equal volume on the day 5, respectively (PBS is an equal volume of cell-free liquid). FIG. 4B shows the in vivo imaging of mice, showing the progression of leukemia in the mice. FIG. 4C shows the statistic of the average weight of mice. FIG. 4D shows the survival curve of mice. FIG. 4E shows the statistic of mouse survival.**P<0.01, *P<0.1.

FIG. 5 shows that B7-H3-CAR-T cells can eliminate tumor cells from AML-transplanted mice. FIG. 5A shows a schematic diagram of the AML transplantation model in NSG mice, wherein three groups of 6- to 8-week-old female NSG mice (PBS, T cell, and B7-H3-CAR-T cell groups), with 3 mice in each group, are injected with 5×10⁵ HEL-luciferase-GFP cells via the tail vein, followed by 2×10⁷ effector cells injected via the tail vein in an equal volume on the second day, respectively (PBS is an equal volume of cell-free liquid). FIG. 5B shows an in vivo imaging image of the mouse, showing the progression of the disease in the mice. FIG. 5C shows the proportion of tumor cells, wherein, after erythrocytes from mouse peripheral blood and bone marrow cells are lysed, cells are incubated with CD45 and CD3 antibodies, and the proportion of tumor cells is detected by flow cytometry**** P<0.0001, *P<0.1. FIG. 5D shows the infiltration of tumor cells, wherein, immunohistochemistry of the spleen and ovarian tissues from the mice by B7-H3 antibody shows that, compared with the B7-H3-CAR-T cell group, there are significant tumor cell infiltration in the PBS and T cell groups at a magnification of ×400 and ×100. n=3.

FIG. 6 shows the safety validation of B7-H3-CAR T cells. FIG. 6A shows the statistics of RBC, WBC, PLT, and HGB after five classification tests of mouse peripheral blood. FIG. 6B shows the HE staining results of mouse tissues, wherein the heart, liver, kidney, and intestinal tissues of mice were taken, HE stained and observed under a microscope to see if there were any lesions in the tissues of mice at a magnification of 100× and 400×. n=3.

FIG. 7 shows the construction of the AML mouse model. FIG. 7A shows the positive rate of luciferase-GFP-HEL cells detected by flow cytometry. FIG. 7B shows a schematic diagram of construction of the mouse AML model, wherein 6- to 8-week-old female NSG mice are injected with 5×10⁵ luciferase-GFP-HEL cells via the tail vein, and then observed. FIG. 7C shows tumor cells in bone marrow, peripheral blood, spleen, liver, and kidney of tumor-bearing mice detected by flow cytometry.

FIG. 8 shows the positive rate of B7-H3-CAR T cells. FIG. 8A shows the B7-H3-CAR-T cell positive rate detected by flow cytometry in the first mouse experiment. FIG. 8B shows the B7-H3-CAR-T cell positive rate detected by flow cytometry in the second mouse experiment.

### EMBODIMENTS

Through extensive and intensive research, the inventor unexpectedly discovered a CAR-T cell targeting B7-H3 for the first time. In vitro functional experiments show that B7-H3-CAR-T cells have significant specific killing effects on B7-H3 positive AML tumor cells. Animal experiments show that B7-H3-CAR-T cells can significantly inhibit the growth of AML tumor cells in mice, significantly prolong the survival of mice, and have significant anti-tumor effects in vivo. It is indicated that the B7-H3-CAR-T cell constructed in the present invention can serve as a novel targeting therapy for AML with great clinical application prospects. On this basis, the present invention has been completed.

### Terms

To make the disclosure easier to understand, some terms are firstly defined. As used in this application, unless expressly stated otherwise herein, each of the following terms shall have the meanings given below. Other definitions are set forth throughout the application.

The term “about” may refer to a value or composition within an acceptable error range for a particular value or composition as determined by those skilled in the art, which will depend in part on how the value or composition is measured or determined.

The term “administering” refers to the physical introduction of a product of the present invention into a subject using any one of various methods and delivery systems known to those skilled in the art, including intravenous, intramuscular, subcutaneous, intraperitoneal, spinal or other parenteral administration, such as by injection or infusion.

The term “antibody” (Ab) may include, but is not limited to, an immunoglobulin that specifically binds an antigen and contains at least two heavy (H) chains and two light (L) chains linked by disulfide bonds, or an antigen binding parts thereof. Each H chain contains a heavy chain variable region (abbreviated herein as VH) and a heavy chain constant region. The heavy chain constant region contains three constant domains, CH1, CH2, and CH3. Each light chain contains a light chain variable region (abbreviated herein as VL) and a light chain constant region. The light chain constant region contains a constant domain CL. The VH and VL regions can be further subdivided into hypervariable regions called complementarity determining regions (CDR), which are interspersed within more conservative regions called framework regions (FR). Each VH and VL contains three CDRs and four FRs, which are arranged from amino terminal to carboxy terminal in the following order: FR1, CDR1, FR2, CDR2, FR3, CDR3, FR4. The variable regions of the heavy and light chains contain a binding domain that interacts with an antigen.

It should be understood that the names of amino acids herein are represented by the internationally accepted single English letter, to which the corresponding three English letter names of amino acid are as follows: Ala (A), Arg (R), Asn (N), Asp (D), Cys (C), Gln (Q), Glu (E), Gly (G), His (H), Ile (I), Leu (L), Lys (K), Met (M), Phe (F), Pro (P), Ser (S), Thr (T), Trp (W), Tyr (Y) and Val (V).

### B7-H3

B7-H3, also known as CD276, was discovered in 2001. It is mainly expressed on the surface of cells, such as activated dendritic cells, monocytes, T cells, B cells, and NK cells. Studies have shown that B7-H3 can stimulate the expansion and killing of T cells, and may selectively stimulate signaling receptors of T cells. At the same time, B7-H3 is a tumor-associated antigen that plays an important role in tumor progression and metastasis. The survival rate of patients without B7-H3 protein expression is higher than that of patients with positive expression. Many studies have shown that B7-H3 is abnormally expressed in human malignancies, such as melanoma, leukemia, and breast and prostate cancer. Studies have discovered that the vast majority of cancer patients exhibit abnormally high expression of B7-H3 in cancer tissue (60-93%), whereas B7-H3 expression in normal healthy tissue is quite limited. B7-H3 is one of the most highly expressed B7 family molecules in AML, with the highest expression in the M3 and M5 subtypes.

It has been reported that B7-H3 is not expressed in immune cells. In addition, most literature regarding B7-H3 has shown that B7-H3 is related to tumor progression and metastasis. More importantly, B7-H3 has been found to be highly expressed in tumor-related stromal cells, fibroblasts and tumor neovascularization. Therefore, treatment targeting B7-H3 is expected to disrupt drug inhibition by the tumor microenvironment, and is a new target that attracts increasing attention in recent years.

Currently, several research groups have constructed B7-H3-CAR-T cells, which shows encouraging tumor cell killing activity in preclinical studies of various solid tumors such as pancreatic cancer, childhood neuroblastoma and osteosarcoma. In the present invention, a B7-H3-CAR-T cell with 4-1BB as a co-stimulatory domain carrying a safety switch, a truncated EGFR molecule (EGFRt), is constructed, which exhibits good cytotoxicity targeting AML tumor cell lines in vitro and in mouse model experiments.

### Chimeric Antigen Receptor

The chimeric antigen receptor (CAR) of the present invention comprises an extracellular domain, a transmembrane domain, and an intracellular domain. The extracellular domain comprises a target-specific binding element (also known as an antigen binding domain). The intracellular domain includes a co-stimulatory signaling region and a ζ chain. The co-stimulatory signaling region refers to a part of the intracellular domain that includes a co-stimulatory molecule. The co-stimulatory molecule is a cell surface molecule required for efficient response of lymphocytes to antigens, rather than a antigen receptor or its ligand.

A linker (or linking peptide) can be incorporated between the extracellular domain and the transmembrane domain of the CAR, or between the cytoplasmic domain and the transmembrane domain of the CAR. As used herein, the term “linker” generally refers to any oligopeptide or polypeptide that plays a role of linking the transmembrane domain to the extracellular domain or the cytoplasmic domain in a polypeptide chain. The linker may comprise 0-300 amino acids, preferably 2-100 amino acids and most preferably 3-50 amino acids.

In a preferred embodiment of the present invention, the extracellular domain of the CAR provided by the present invention comprises an antigen binding domain targeting B7-H3. When the CAR of the present invention is expressed in T cell, antigen recognition can be performed based on antigen binding specificity. When the CAR binds to its associated antigen, it affects tumor cell, causing tumor cell to fail to grow, to death or to be affected otherwise, causing the patient's tumor burden to shrink or eliminate. The antigen binding domain is preferably fused to one or more intracellular domains of the co-stimulatory molecule and the chain. Preferably, the antigen binding domain is fused with an intracellular domain of a combination of an ICOS and/or 4-1BB signaling domain and a CD3ζ signaling domain.

As used herein, both “antigen binding domain” and “single chain antibody fragment” refer to a Fab fragment, a Fab′ fragment, a F(ab′)₂ fragment, or a single Fv fragment having antigen binding activity. A Fv antibody contains a heavy chain variable region and a light chain variable region of the antibody, but does not have a constant region, and has the smallest antibody fragment with all antigen binding sites. Generally, a Fv antibody also contain a peptide junction between VH and VL domains and can form the structures required for antigen binding. The antigen binding domain is usually a scFv (single chain variable fragment). The size of a scFv is generally ⅙ of that of a complete antibody. Single chain antibodies are preferably an amino acid chain sequence encoded by a single nucleotide chain. As a preferred method of the present invention, the scFv specifically recognizes B7-H3.

As for the hinge region and the transmembrane region (transmembrane domain), the CAR can be designed to comprise a transmembrane domain fused to the extracellular domain of the CAR. In one embodiment, a transmembrane domain that is naturally associated with one of the domains in the CAR is used. In some embodiments, transmembrane domains may be selected or modified by amino acid substitutions to avoid binding such domains to the transmembrane domain of the same or different surface membrane proteins, thereby minimizing the interaction with other members of the receptor complexes.

The intracellular domain in the CAR of the present invention comprises the signaling domain of 4-1BB and the signaling domain of CD3ζ.

### Vector

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically.

The present invention also provides vectors in which the expression cassette of the present invention is inserted. Vectors derived from retroviruses such as the lentivirus are suitable tools to achieve long-term gene transfer since they allow long-term, stable integration of a transgene and its propagation in daughter cells. Lentiviral vectors have the advantage over vectors derived from onco-retroviruses such as murine leukemia viruses in that they can transduce non-proliferating cells, such as hepatocytes. They also have the advantage of low immunogenicity.

In brief, the expression cassette or nucleic acid sequence of the present invention is typically and operably linked to a promoter, and incorporated into an expression vector. The vector is suitable for replication and integration in eukaryotes. A typical cloning vector contains transcription and translation terminators, an initiation sequence, and promoters useful for regulation of the expression of the desired nucleic acid sequence.

The expression construct of the present invention may also be used for nucleic acid immune and gene therapy, using standard gene delivery protocols. Methods for gene delivery are known in the art, see, e.g., U.S. Pat. Nos. 5,399,346, 5,580,859, and 5,589,466, which are incorporated by reference herein in entirety. In another embodiment, the present invention provides a gene therapy vector.

The nucleic acid can be cloned into various vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al, (2001, Molecular Cloning: A Laboratory Manual, Cold Spring Harbor Laboratory, New York), and in other virology and molecular biology manuals. Viruses which are useful as vectors include but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for transferring a gene into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentiviral vectors are used.

Additional promoter elements, e.g., enhancers, can regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the present invention should not be limited to the use of constitutive promoters. Inducible promoters are also contemplated as part of the present invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionein promoter, a glucocorticoid promoter, a progesterone promoter, and a tetracycline promoter.

In order to assess the expression of a CAR polypeptide or portions thereof, the expression vector to be introduced into a cell may also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In a preferred embodiment of the present invention, the vector is a lentiviral vector.

### Therapeutic Application of CAR-T

The present invention comprises therapeutic applications by using cells (e.g., T cells) transduced with a lentiviral vector (LV) encoding the expression cassette of the present invention. The transduced T cells can target the tumor cell marker B7-H3, synergistically activate T cells, and cause T cell immune responses, thereby significantly increasing the killing efficiency against tumor cells.

Therefore, the present invention also provides a method for stimulating a T cell-mediated immune response to a target cell population or tissue in a mammal comprising a step of administering to the mammal a CAR-T cell of the present invention.

In one embodiment, the present invention comprises a cell therapy, wherein T cells from autologous patient (or heterologous donor) are isolated, activated and genetically modified to generate CAR-T cells, and then injected into the same patient. The probability of graft-versus-host-disease in the way is extremely low, and antigens are recognized by T cells in a non-MHC-restricted manner. In addition, one CAR-T can treat all cancers that express the antigen. Unlike antibody therapy, CAR-T cells are able to replicate in vivo and result in long-term persistence that can lead to sustained tumor control.

In one embodiment, the CAR-T cells of the present invention can undergo robust in vivo T cell expansion and can persist for an extended period. In addition, the CAR mediated immune response may be part of an adoptive immunotherapy approach in which CAR-modified T cells induce an immune response specific to the antigen binding moiety in the CAR. For example, an anti-B7-H3 CAR-T cell elicits an immune response specific against cells expressing B7-H3.

Although the data disclosed herein specifically disclose lentiviral vector comprising anti-B7-H3 scFv, hinge and transmembrane domains, and 4-1BB and CD3ζ signaling domains, it is contemplated that the present invention include any number of variations for each of the components of the construct as described elsewhere herein.

Cancers that may be treated include tumors that have not been vascularized or have not yet been vascularized, and tumors that are vascularized. Cancers may include non-solid tumors (such as hematological tumors, for example, leukemias and lymphomas) or solid tumors. Types of cancers to be treated with the CARs of the present invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblasts, promyeiocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors (such as sarcomas and carcinomas) include fibrosarcoma, myxosarcoma, liposarcoma, mesothelioma, malignant lymphoma, pancreatic cancer and ovarian cancer.

In a preferred embodiment, the CAR-T cell of the present invention can be used for treating acute myeloid leukemia.

The CAR-modified T cells of the present invention may also serve as a type of vaccine for ex vivo immunization and/or in vivo therapy in a mammal. Preferably, the mammal is a human.

With respect to ex vivo immunization, at least one of the following occurs in vitro prior to administering the cells into a mammal: i) expanding the cells, ii) introducing a nucleic acid encoding a CAR to the cells, and/or iii) cryopreservation of the cells.

Ex vivo procedures are well known in the art and are discussed more fully below. Briefly, cells are isolated from a mammal (preferably a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

In addition to using a cell-based vaccine in terms of ex vivo immunization, the present invention also provides compositions and methods for in vivo immunization to elicit an immune response directed against an antigen in a patient.

The present invention provides a method for treating tumors comprising administering to a subject in need thereof, a therapeutically effective amount of the CAR-modified T cells of the present invention.

The CAR-modified T cells of the present invention may be administered either alone, or as a pharmaceutical composition in combination with diluents and/or with other components such as IL-2, IL-17 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a patient subcutaneously, intradermaliy, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatments, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the present invention may be used in combination with chemotherapy, radiation, immunosuppressive agents, such as cyclosporin, azathioprine, methotrexate, mycophenolate, and FK506, antibodies, or other immunotherapeutic agents. In a further embodiment, the cell compositions of the present invention are administered to a patient in conjunction with (e.g., before, simultaneously or following) bone marrow transplantation, or the use of chemotherapy agents such as, fludarabine, external-beam radiation therapy (XRT), cyclophosphamide. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to practices in the field. In general, 1×10⁶ to 1×10¹¹ of the modified T cells of the present invention can be applied to patients by means of, for example, intravenous infusion in each treatment or each course of treatment.

### The Main Advantages of the Present Invention Include:

(a) The B7-H3-CAR-T cell constructed in the present invention has significant in vitro anti-tumor effect on AML cell lines.
(b) The B7-H3-CAR-T cell constructed in the present invention has significant in vivo anti-tumor effect on AML cell line HEL tumor-bearing mice.
(c) The results of mouse experiments indicate that the B7-H3-CAR-T cell constructed in the present invention has good safety.

The invention is further illustrated below in conjunction with specific embodiments. It should be understood that the examples are not intended to limit the scope of the invention. The experimental methods in the following examples which do not specify the specific conditions are usually in accordance with conventional conditions, such as conditions described in Sambrook et al., Molecular Cloning: Laboratory Manual (New York: Cold Spring Harbor Laboratory Press, 1989), or in accordance with the conditions recommended by the manufacturer. Unless otherwise stated, percentages and parts are by weight.

### General Materials and Methods:

### 1. Cells

KG-1, MOLM-16 and Jurkat cells were purchased from American Type Culture Center (ATCC; Manassas, VA, USA). HEL, THP-1, HL-60 and AML5 cell lines were donated by laboratory of Professor Zhao Yun, Tang Zhongying hematology research center of Suzhou University.

RP-MI1640 medium supplemented with 10% FBS was used as medium of HEL cells, Jurkat cells and AML5 cells. IMDM medium supplemented with 10% FBS was used as medium of KG-1 cells. Peripheral blood mononuclear cells (PBMCs) were extracted from normal human peripheral blood by Ficoll method. T cells were activated by Transact, and cultured in TexMACSTMGMP medium supplemented with IL-7 and IL-15. All cells were cultured in a cell incubator containing 5% CO₂ at 37° C. After being activated for 48 h in vitro, the extracted T cells were transfected. The toxicity test was carried out when the cell proliferated to a sufficient number.

### 2. Construction of CAR

Second generation CAR is constructed in the present application, wherein the sequence of B7-H3-scFv was constructed into the lentiviral vector, and B7-H3-scFv, CD8 hinge and transmembrane domain, 41BB intracellular sequence and CD3ζ sequence are successively comprised.

3. Packaging of Virus and Transfection of T/Jurkat Cells Lentiviruses were prepared according to conventional methods in the prior art. After the T cells were activated for 48 h, Jurkat cells were placed on a 48-well plate (5×10⁵ cells/well). 50 μL of virus was added, and the final system was 100 μl. After transfection for 15 h, 1000 μl of medium was added for cultivation.

### 4. Flow Cytometry Analysis

Six AML cell strains, which were AML5, KG-1, HEL, HL-60, THP-1 and MOLM-16, were collected. After centrifugation, the supernatant was discarded and cells were washed once with PBS of pH=7.4. After incubation with FITC-fluorescent B7-H3 antibody (1:100), the cells were washed twice with PBS. The expression of B7-H3 on the cell surface was detected by flow cytometry.

About 2×10⁶ cells of cultured B7-H3-CAR Jurkat cells, Jurkat cells, B7-H3-CAR T cells, and T cells were taken out respectively, co-incubated with 100 μl of EGFR antibody diluted at 1:1000 (final concentration of the antibody: 1 μg/ml) at 37° C. for 20 minutes, and washed twice with PBS. Jurkat cells or T cells were used as control cells. The B7-H3-CAR positive rates of B7-H3-CAR Jurkat and B7-H3-CAR T cells were analyzed and detected by using flow cytometry.

Jurkat cells and B7-H3-CAR Jurkat cells co-incubated with KG-1, HEL, and AML5 cells were incubated with CD25-APC and CD69-APC antibodies for 20 minutes, respectively. Then the expression of CD25 and CD69 was detected by using flow cytometry.

### 5. WESTERN Detection

About 1×10⁶ T cells and B7-H3-CAR T cells were taken respectively and lysed, and the products were separated by SDS-PAGE and transferred to the polyvinylidene fluoride (PVDF) membrane (micropores). The products were blocked with 5% skim milk for 1 hour and then incubated in mice-anti-human CD3ζ antibody at 4° C. overnight. Subsequently, after incubation with HRP-sheep-anti-mouse IgG at room temperature for 2 hours, the membrane was added with developer and exposed in the dark room for development.

### 6. Detection of the Toxic Effect of B7-H3 CAR T Cells on Tumor Cells

1×10⁵ KG-1, HEL, and AML5 tumor cell were taken and centrifuged, incubated with 100 μl of CFSE (1:1000) at 37° C. for 15 minutes, washed twice with PBS and added together with the effector cells at a ratio of 1:1 to a 24 well, and the liquid was supplemented to 2 ml. After 48 hours, the cells were taken out and centrifuged. The supernatant was taken for cytokine detection. The precipitate was washed once with PBS and the apoptotic tumor cell proportion was detected using the Annexin V-APC/7-AAD apoptosis detection kit (LK-AP105-100; Lianke Biotech, Hangzhou, China, http;//www.liankebio.com).

### 7. Detection of Cytokines

The amount of cytokines produced by T cells and B7-H3-CAR T cells in the supernatant after killing was detected by using the cytometric bead array (CBA). Human Granzyme B CBA Flex Set D7 Kit (Cat. No. #560304), Human TNF Flex Set D9 Kit (Cat. No. #558273), Human IL-2 Flex Set A4 Kit (Cat. No. #558270) and Human IFN-γ CBA Flex Set E7 Kit (Cat. No. #558269) purchased from BD Company were used for the detection.

### 8. AML Mouse Model

Models were constructed using 6-8 week NOD-Prkdcscidll2rgtm1/Bcgen(B-NSG) female mice purchased from Biocytogen Company. Mouse were injected with 5×10⁵ HEL-luciferase-GFP cells through tail vein, and the tumor burden was detected using the IVIS spectrum system after 2 or 5 days. The mice were intraperitoneally injected with 150 mg/kg D-luciferin substrate, and the biofluorescence thereof was recorded after 10 minutes. 1×10⁷ effector cells were injected in an equal volume into the tail vein of each mouse (PBS is an equal volume of cell-free liquid). 100 μL of peripheral blood, femoral bone marrow, and ground organs were taken for erythrocyte lysis, then incubated with anti-human CD45 antibody for flow cytometry analysis to detect tumor cells.

### 9. Immunohistochemical Analysis

Mice were sacrificed and the spleen and ovarian tissues were taken, embedded in paraffin and sliced (as thick as 3 m) for immunohistochemical analysis. The paraffin slices were dewaxed and rehydrated, and immersed in 0.01M citrate buffer solution, retrieved with microwave for 5 minutes, and then sealed with a methanol solution containing 0.3% hydrogen peroxide. The slices were stained with an antibody targeting B7-H3 (diluted at 1:300) followed by a biotin-labeled secondary antibody. Finally, DAB chromogenic solution was added. The infiltration of tumor cells in the tissues was observed. Haematoxylin and eosin (HE) staining was performed after the paraffin sections were dewaxed and rehydrated, to observe the pathological changes in tumor tissues.

### 10. Statistical Analysis

Graphpad prism 5 software was used. T-tests were used for the comparison between two groups. ****P<0.0001, ***P<0.001, *P<0.01 and *P<0.05 represent statistically significant differences.

### Example 1 Expression of B7-H3 in AML Cell Lines

Six AML cell lines were selected to detect their B7-H3 expression by flow cytometry.

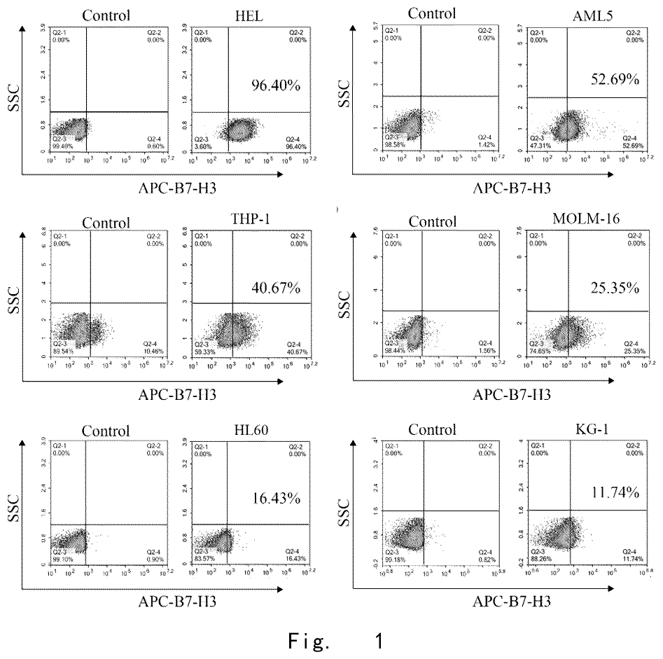

The result of flow cytometry is shown in FIG. 1, wherein the expression rates of B7-H3 are 96.4% in HEL cells, 52.69% in AML5 cells, 40.67% in THP-1 cells, 25.35% in MOLM-16 cells, 16.43% in HL-60 cells, and 11.74% in KG-1 cells. Finally, HEL, AML5, and KG-1 cells (with high, medium, and low expression of B7-H3) were selected for later experiments.

### Example 2 Construction and Specificity Verification of B7-H3-CAR

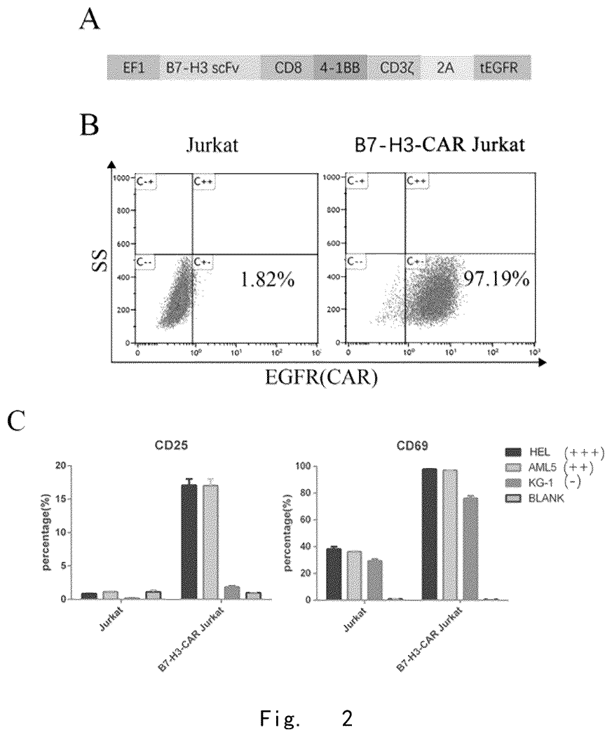

Firstly, a vector of CAR targeting B7-H3 was constructed, wherein the CAR is a second generation CAR containing a B7-H3 single chain antibody and a 41BB co-stimulatory domain. EGFR was co-expressed as a detection label for the CAR. The specific structure is shown in FIG. 2A. The amino acid is shown in SEQ ID NO. 3. Lentivirus were packaged and used transfected into Jurkat cells to construct B7-H3-CAR-Jurkat cells that can stably and highly express B7-H3-CAR. The positive rate of CAR in B7-H3-CAR-Jurkat cells detected by flow cytometry was as high as 97.19% (FIG. 2B).

Jurkat cells before transfection and B7-H3-CAR Jurkat cells after transfection were co-incubated with HEL, AML5, and KG-1 cells at an E:T ratio of 1:1 for 48 hours. It was detected that, after co-incubation with tumor cells expressing high B7-H3 antigen, the expression of both CD25 and CD69 on the surface of B7-H3-CAR Jurkat cells are higher than that of untransfected Jurkat cells. However, after co-incubation with tumor cells expressing low B7-H3 antigen, there is no significant difference in the expression of CD25 and CD69 on the surface of B7-H3-CAR Jurkat cells and Jurkat cells (FIG. 2C).

The aforementioned results indicate that the B7-H3-CAR-Jurkat cells constructed in the present invention can be specifically activated by B7-H3-positive tumor cells.

### Example 3: In Vitro Killing of B7-H3-CAR T Cells on B7-H3 Positive Tumor Cells

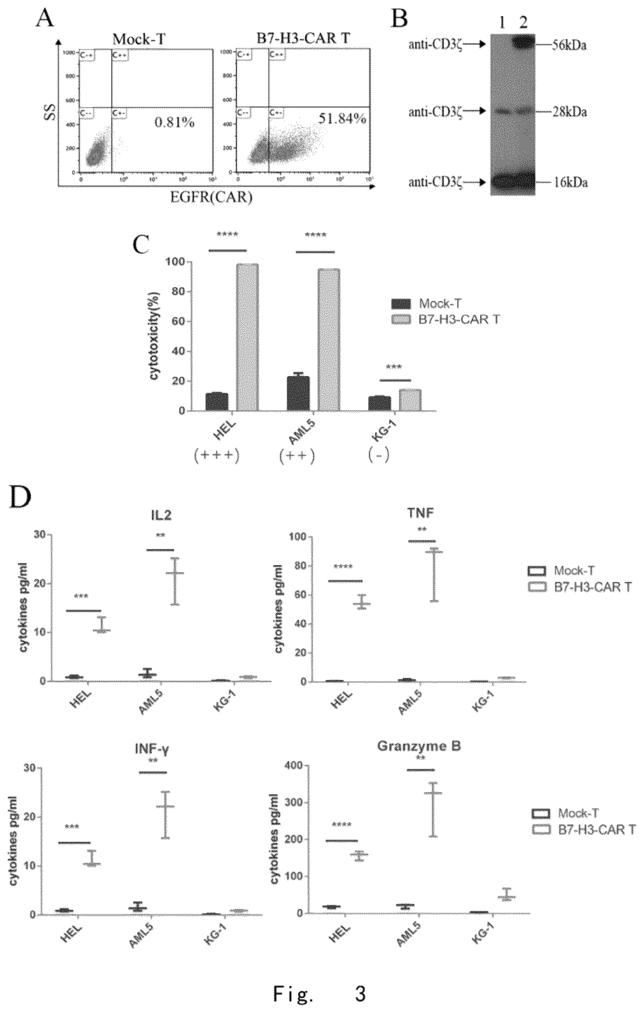

To further demonstrate the in vitro biological function of the B7-H3-CAR constructed in Example 2, BB7-H3-CAR-T cells were prepared, and the positive rate was approximately 51.84% as determined by flow cytometry (FIG. 3A). Western blotting indicates the expression of B7-H3-CAR fusion protein in the B7-H3-CAR T cells (FIG. 3B), which includes two 16 kD and 28 kD endogenous CD3ζ (lane 1 and lane 2) expressed by all T cells, and one exogenous CD3ζ (56 kD, lane 2) expressed by CAR-T cells.

After co-incubating T cells and prepared B7-H3-CAR T cells with HEL, AML5, and KG-1 cells at an E:T ratio of 1:1 for 48 hours, the apoptosis of tumor cells was detected. The results show that, compared with T cells, B7-H3-CAR-T cells have a good cytotoxic effect on tumor cells with high and moderate expression of B7-H3. However, there was no significant difference in tumor cells with low B7-H3 expression. This demonstrates that B7-H3-CAR-T cells have a strong cytotoxic effect on AML cells expressing B7-H3 (FIG. 3C).

Subsequently, the secretion of cytokines in the supernatant was detected, and the results were consistent with the cytotoxicity results. The cytokine secretions of B7-H3-CAR-T cells co-incubated with tumor cells with high and moderate expression of B7-H3 were significantly higher than those of other groups (FIG. 3D). The results further suggests that the B7-H3-CAR-T cells constructed in the present invention can specifically kill AML tumor cells expressing B7-H3.

### Example 4: B7-H3-CAR T Cells can Prolong the Survival of Tumor Bearing Mice

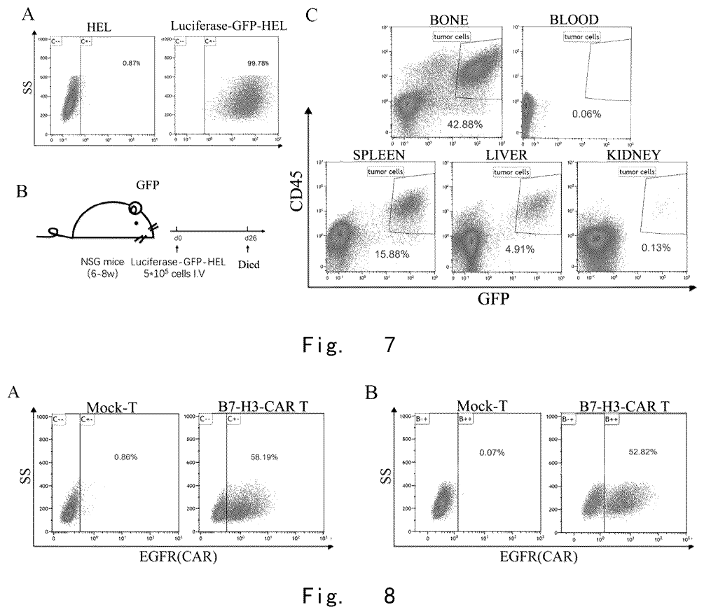

To further evaluate the anti-tumor activity of B7-H3-CAR T cells in vivo, luciferase-GFP-HEL cells were successfully constructed (FIG. 7A).

First, an AML mouse tumor model was established. 6- to 8-week-old NSG female mice were injected with 5×10⁵ luciferase-GFP-HEL cells via the tail vein. Mice were observed, and after approximately 20 days, depression, hind limb paralysis, emaciation, and weight loss occurred successively (FIG. 7B). After natural death, bone marrow, peripheral blood, spleen, liver and kidney of mice were collected for flow cytometry analysis of tumor cell proportion (FIG. 7C). Experiment shows that mice injected with 5×10⁵ tumor cells via the tail vein could be used to successfully establish the AML model.

Luciferase-GFP-HEL cells were injected into the tail vein of 12 B-NSG mice (divided into 3 groups of 4 in each group) (FIG. 4A). Each mouse was injected with 5×10⁵ cells. On the fifth day, in vivo imaging of mice detected a high proportion of tumor cells in mice, which is equivalent to a model of advanced AML cases. Thereafter, the same amount of PBS/T/B7-H3-CAR-T (positive rate is approximately 58.19%, as shown FIG. 8A) cells were injected via the tail vein. The survival of mice was observed, during which tumor burden in the mice was detected using in vivo imaging every few days. The in vivo imaging shows that the tumor progression in the mice in the B7-H3-CAR-T cell group was significantly weaker than in the other two groups (FIG. 4B). The average weight of mice in the PBS group and T cell group had already sharply decreased around day 30, with consecutive deaths, while the weight of mice in the B7-H3 group was still nearly flat (FIG. 4C). After all the mice died, their survival period was counted. The results showed that the B7-H3-CAR T cell group mice had a significantly longer survival than those of mice from the PBS group and T cell group, which has a statistical difference (FIG. 4D, FIG. 4E). This indicates that B7-H3-CAR T cells can effectively inhibit the growth of tumor cells in tumor-bearing mice, and have significant in vivo anti-tumor effects on B7-H3 positive AML cells.

### Example 5: B7-H3-CAR T Cells can Reduce Tumor Burden in Peripheral Blood and Bone Marrow of Tumor-Bearing Mice

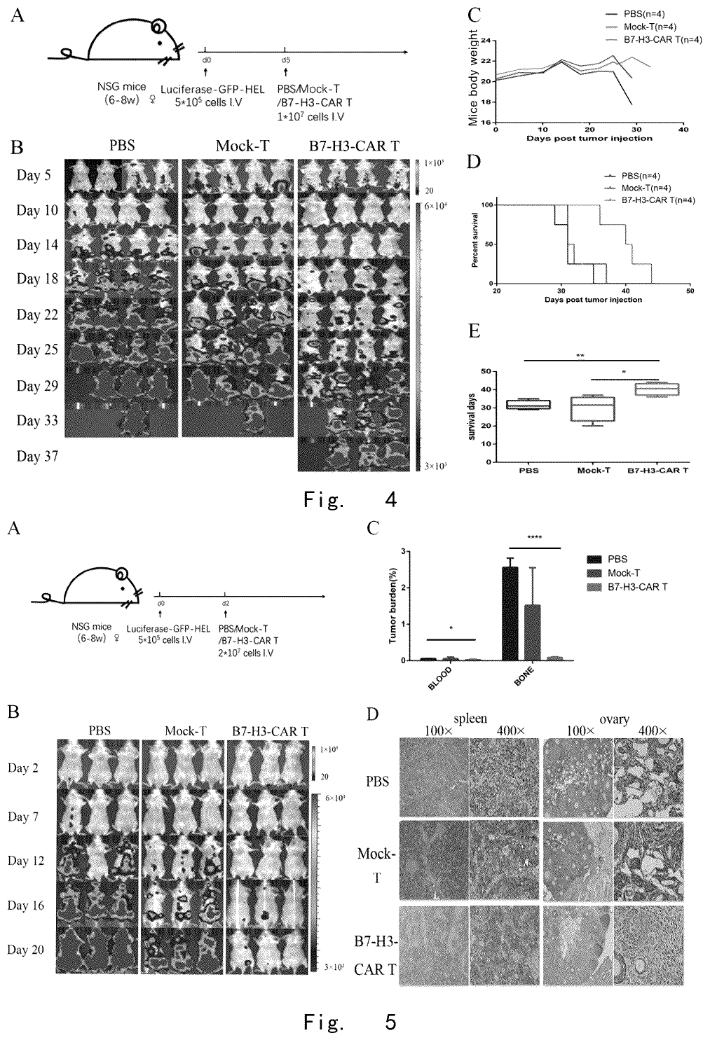

In order to further demonstrate the in vivo anti-tumor effect of B7-H3-CAR T cells, another batch of mouse models were established (FIG. 5A). On the second day after the injection of tumor cells, in vivo imaging shows the tumor cell infiltration into the bone marrow, indicating successful modeling. Then 2×10⁷ T cells and B7-H3-CAR T cells (CAR positive rate is 52.82%, FIG. 8B) were injected into mice, respectively. After that, in vivo imaging was performed every 4-5 days. The results show that, compared with mice from PBS group and T cell group, the tumor growth of mice from B7-H3-CAR T cell group was significantly inhibited (FIG. 5B).

At day 20, mice from PBS group exhibited symptoms such as weight loss, mental exhaustion, and quadriplegia. Therefore, all mice were sacrificed, and peripheral blood and bone marrow cells thereof were taken. The proportion of tumor cells was detected using flow cytometry (FIG. 5C). The results show that the tumor burden of mice from B7-H3-CAR T cell group was significantly lower than that of the other two groups of mice.

Immunohistochemical analysis of the spleen and ovarian tissues of mice show significant tumor cell infiltration in the tissues of mice from PBS and T cell groups, while there was almost no or only a few tumor cell infiltration in the tissues of the B7-H3 group mice. Therefore, it is demonstrated that B7-H3-CAR T cells can effectively reduce the development of tumor cells in mice.

### Example 6: B7-H3-CAR T Exhibit No Toxicity to Normal Cells

The scFv sequence used in the present invention has high affinity, faster dissociation constant, higher killing activity, and safety. The sequence can simultaneously recognize mouse B7-H3 molecules. The safety of the sequence can be demonstrated by detecting the activity of normal cells in mice.

Peripheral blood routine tests were performed on mice from blank group, PBS group, T cell group, and B7-H3-CAR T cell group (mouse model of Example 5). The results show that there was no significant difference between the white blood cells, red blood cells, platelets, or hemoglobin of B7-H3-CAR T cell group mice and the blank group mice (FIG. 5D). Moreover, HE staining results also show that, the important organs such as heart, liver, kidney, and intestine of mice from B7-H3-CAR T cell group did not show any pathological changes compared with those of healthy mice. Therefore, the above experiment demonstrates that the B7-H3-CAR T cell developed in the present invention is a highly safe CAR-T product.

### Discussion

AML is a refractory invasive hematopoietic stem cell malignancy. In the past decade, the life expectancy of patients with AML has improved due to the development of new treatment methods. However, AML remains one of the most deadly and difficult to treat malignant cancers due to its high drug resistance. Moreover, patients with AML have limited treatment options and a poor prognosis. Chemotherapy is not curative, and patients are prone to relapse. Therefore, new treatment methods are urgently needed. CAR-T therapy has made great progress in both haematologic and solid tumors, and is considered to be one of the most promising tumor treatment methods.

At present, the CAR-T treatment of AML requires bone marrow clearance before treatment in most of the patients because most of the targets are also highly expressed in normal myeloid cells, which greatly complicates the treatment procedure and increases many unnecessary risks. Therefore, it is important to find an antigen that is selectively expressed in malignant cells but lacking in normal cells. Statistics show that approximately 40% of patients with AML express B7-H3. Moreover, some studies have shown B7-H3 expression in >50% of cells in 31 pancreatic tumor specimens, but not in normal pancreatic tissue specimens. B7-H3 expression is regulated by RNA transcription and is a surface immunoregulatory glycoprotein that inhibits natural killer cells and T cells. Although B7-H3 transcripts are widely expressed in human solid tumors and normal tissues, the B7-H3 protein is preferentially expressed only in tumor tissues. Some scholars have also pointed out that B7-H3 is expressed in only a few tissues and cells, including activated lymphocytes and tumor cells. Therefore, although B7-H3 is expressed in part of AML, it is an excellent therapeutic target for AML because it does not cause toxicity to the hematopoietic system.

Previous studies have shown that T cells is not sufficient to combat cunning tumor cells solely, but the B7-H3-CAR T cells of the present invention can effectively and specifically kill AML cells expressing B7-H3 (FIG. 3C). B7-H3-CAR T cells also have a significant tumor elimination effect in mice. In vivo imaging detection show that the tumor cells in mice from B7-H3-CAR T cell group were significantly reduced than those of mice from the PBS group and T cell group (FIGS. 4B, 5B). Moreover, even in AML, a disease with a particularly rapid progression, B7-H3-CAR T cells can significantly prolong the survival of mice (FIGS. 4D, 4E) and reduce the proportion of tumor cells in peripheral blood, bone marrow, and tissues (FIGS. 5C, 5D). Therefore, the B7-H3-CAR T cell of the present invention can be used for the treatment of patients with refractory relapsed AML expressing B7-H3 antigen.

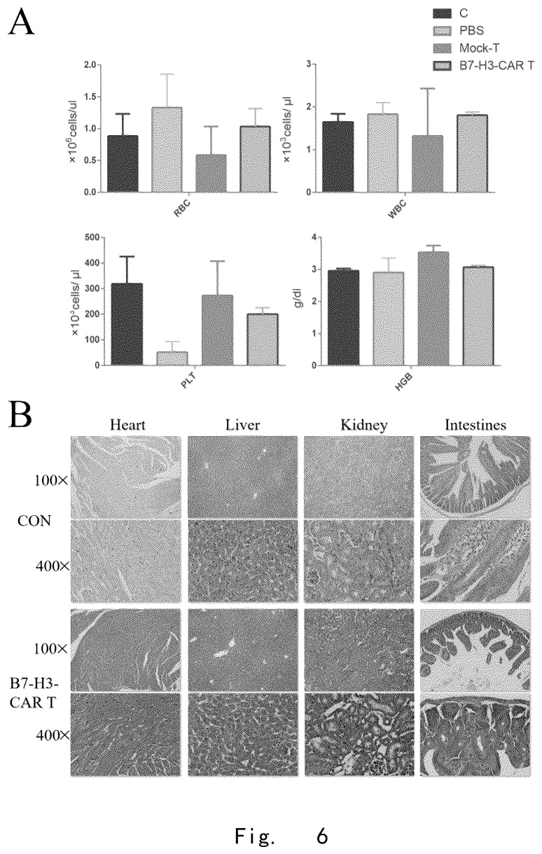

In addition, safety verification was also conducted by detecting whether B7-H3-CAR T cell causes off-target toxicity to normal cells in mice. Peripheral blood routine tests show that B7-H3-CAR T cell did not have toxic effects on normal cells in mice (FIG. 6A). Moreover, HE staining results also show that B7-H3-CAR T cells did not injure important organs in mice (FIG. 6B). Therefore, the B7-H3-CAR T cell of the present invention is a safe CAR-T product.

In summary, the present invention not only demonstrates that the B7-H3-CAR T cell can effectively and specifically eliminate AML tumor cells expressing B7-H3 antigen in vitro and in vivo, but also demonstrates its good safety, laying a foundation for the clinical use of B7-H3-CAR T cells for the treatment of AML. Compared with the most commonly used CD33-CAR T and CD123-CAR T cells which require prior bone marrow clearance before administration, B7-H3-CAR T cell not only has the possibility of prolonging the survival of relapsed refractory AML patients, but also simplifies the difficulty of administration of CAR T treatment in clinical practice and reduces risk in the patient.

All documents mentioned in the present invention are incorporated by reference herein as if each document were incorporated separately by reference. Furthermore, it should be understood that after reading the foregoing teachings of the invention, various changes or modifications may be made to the invention by those skilled in the art and that these equivalents are equally within the scope of the claims appended to this application.

1. A chimeric antigen receptor (CAR), wherein an antigen binding domain of the chimeric antigen receptor comprises a heavy chain variable region and a light chain variable region,
the heavy chain variable region comprises the following complementarity determining regions (CDRs):
a CDR1 as shown in SEQ ID NO: 6,
a CDR2 as shown in SEQ ID NO: 7, and
a CDR3 as shown in SEQ ID NO: 8;
and the light chain variable region comprises the following complementarity determining regions (CDRs):
a CDR1′ as shown in SEQ ID NO: 9,
a CDR2′ as shown in SEQ ID NO: 10, and
a CDR3′ as shown in SEQ ID NO: 11.

2. The CAR according to claim 1, wherein the structure of the chimeric antigen receptor is shown in the following Formula III:
L-scFv-H-TM-C-CD3ζ (III)
wherein,
L is absent or a signal peptide sequence;
scFv is a scFv targeting B7-H3;
H is a hinge region;
TM is a transmembrane domain;
C is a co-stimulatory signaling molecule;
CD3ζ is a cytoplasmic signal transduction sequence derived from CD3ζ.

3. A nucleic acid molecule encoding the chimeric antigen receptor (CAR) according to claim 1.

4. A vector comprising the nucleic acid molecule according to claim 3.

5. An engineered immune cell expressing the CAR according to claim 1.

6. The immune cell according to claim 5, wherein the cell is a T cell or NK cell.

7. A formulation comprising the immune cell according to claim 5, and a pharmaceutically acceptable carrier.

8-9. (canceled)

10. A method for preparing an engineered immune cell expressing the CAR according to claim 1, which comprises following steps:
(a) providing an immune cell to be modified; and
(b) transducing the nucleic acid molecule encoding the CAR according to claim 1 into the immune cell, thereby obtaining the engineered immune cell.

11. The CAR according to claim 1, wherein the antigen binding domain comprises a heavy chain variable region of antibody as shown in SEQ ID NO: 1, and a light chain variable region of antibody as shown in SEQ ID NO: 2.

12. The CAR according to claim 2, wherein L is a signal peptide derived from macrophage colony stimulating factor.

13. The CAR according to claim 2, wherein H is a hinge derived from CD8.

14. The CAR according to claim 2, wherein TM is a transmembrane region derived from CD8.

15. The CAR according to claim 2, wherein C is a co-stimulatory signaling molecule derived from 4-1BB.

16. The CAR according to claim 1, which further comprises a cell suicide element.

17. The CAR according to claim 1, of which the amino acid sequence is shown in SEQ ID NO: 3.

18. A method for treating a disease, which comprises: administering an appropriate amount of the immune cell according to claim 5 to a subject in need of treatment.

19. The method according to claim 18, wherein the disease is a cancer or tumor.

20. The method according to claim 18, wherein the tumor is a B7-H3-positive tumor.

21. The method according to claim 18, wherein the disease is acute myeloid leukemia (AML).
